(19) United States
(12) Patent Application Publication
(10) Pub. No.: US 2005/0208586 A1
(43) Pub. Date: Sep. 22, 2005

(54) Using complement component C1q derived molecules as tracers for fluorescence polarization assays

(76) Inventor: Shanxiang Shen, Bethesda, MD (US)

Correspondence Address: SHANXIANG SHEN, 17 ORCHAR WAY S., POTOMAC, MD, 20854, US

(21) Appl. No.: 10/803,246

(22) Filed: Mar. 18, 2004

(51) Int. Cl. G01N 33/53; C07K 16/46

(52) U.S. Cl. 435/7.1; 530/391.1

(57) ABSTRACT

The present invention relates to polypeptides or non-polypeptides derived from C1q, a subunit of the first complement component molecule C1. These molecules bind to the C1q receptor on the Fc domain of an antibody in immune complexes (also called antigen-antibody complexes or aggregated immunoglobulins), but do not bind to free immunoglobulins. These complement component derived molecules may be used as tracer molecules for fluorescence polarization (FP). The present invention also relates to materials and methods of producing molecules for use in FP. The present invention has many applications in the areas of biosensor development for proteomics research, protein expression profiling, drug discovery, diagnosis and prognosis, monitoring therapeutic effects, environmental survey, and bio-defense.

### FIELD OF THE INVENTION

This invention relates to a new detection method for immune complexes utilizing principles of fluorescence polarization and tracer molecules derived from a subunit, C1q, of the first complement component molecule, C1.

### SUMMARY OF THE INVENTION

The present invention relates to polypeptide molecules that are derived from a subunit, C1q, of the first complement component molecule, C1. The present invention also relates to non-polypeptide molecules that can mimic desired binding behavior of C1q. These molecules bind to the C1q receptor on the constant region of an antibody in immune complexes, but do not bind to free immunoglobulins, and may be used as tracer molecules in fluorescence polarization (FP) assays. The present invention also relates to materials and methods of producing these molecules for using in FP assays. The present invention has many applications in the areas of assay and biosensor development for proteomics research, protein expression profiling, drug discovery, diagnosis and prognosis, monitoring therapeutic effects, environmental survey, and bio-defense.

### DETAILED DESCRIPTION OF THE INVENTION

Since the unveiling of the entire human genome sequence in the year 2000, genomic research has progressed to the next logical step, proteomics, which is large-scale research of protein functions. One of the most important fields in proteomics research is the detection of binding between a protein and its corresponding ligands. One such example is the specific interaction between an antibody and its antigen to form an immune complex, (the antibody then also called bound antibody or aggregated immunoglobulin). The application of the knowledge of antibody-antigen interactions is extremely valuable in bio-medical research and has numerous practical applications including drug discovery and bio-defense.

Antibodies are molecules produced by vertebrates' immune systems. One function of antibodies is to recognize their corresponding ligands (i.e., antigens), with high specificity. Antigens can be any molecule from a living organism including plants and animals as well as organic or inorganic compounds. In proteomics research, antibody-antigen reactions are widely utilized in protein expression profiling and discovering therapeutic antibodies. They are also frequently used for detecting pathogens, cancer, and other markers in disease diagnosis and prognosis.

Most existing methods for detecting antibody-antigen reactions involve immobilization of a capturing molecule, such as an antibody, on a solid surface. Immobilization is a time consuming process and a complete assay requires many washing steps. Also, immobilization often causes conformational changes or denaturation of protein molecules that consequently affect the accuracy of the process. Furthermore, non-specific binding of non-target proteins or compounds to the solid surface is a significant problem for many solid phase assays.

Fluorescence polarization (FP) refers to the polarization of fluorescent light emitted by fluorophores and is an alternative to existing solid phase assays. Assays based on the principals of FP are conducted in a homogenous liquid phase and no molecule immobilization is involved. When using fluorescence polarization techniques, a fluorescent molecule is excited by polarized light and will emit fluorescence that has a degree of polarization inversely proportional to the molecule's rate of rotation. Small fluorescent molecules rotate relatively more quickly and therefore have lower degree of polarization, while large molecules rotate relatively more slowly and have higher degree of polarization. When a small fluorescent molecule binds to a large non-fluorescent molecule, the complex rotates slower than the unbound small fluorescent molecule. Therefore, the degree of polarization increases. Fluorescence polarization therefore provides a direct readout of the extent of small tracer binding to macromolecules such as proteins and nucleic acids. Tracers used in fluorescence polarization assays can be conjugates of polypeptides and non-polypeptide chemical compounds with fluorescent molecules. Because polarization is a general property of fluorescent molecules, polarization-based readouts are less dye-dependent and less susceptible to environmental interferences such as pH changes than those based on fluorescent intensity.

Fluorescence polarization measurements provide information on molecular orientation and mobility, and the processes that modulate them, including receptor-ligand interactions, proteolysis, protein-DNA interactions, membrane fluidity and muscle contraction. Fluorescence polarization measurements have long been a valuable biophysical research tool for investigating processes such as membrane lipid mobility, myosin reorientation and protein-protein interactions at the molecular level.

One practical application of FP principles is fluorescence polarization immunoassays (FPIA). Immunoassays that have been developed and used extensively for clinical diagnostics represent the largest group of bio-analytical applications. If an antibody specific for the small fluorescent tracer molecule is added, they combine to become a large molecule that rotates much slower. The slower rotation of the antibody-antigen complex causes it to emit fluorescence in the same polarized plane as the incident light. Measurement of the amount of polarized fluorescence emitted, gives an estimate of the quantity of antibody-antigen complexes in the solution. FPIA is used to measure patient serum drug levels such as phenobarbitol, Primidone, digoxin, benzodiazapines, tricyclic antidepressants, and cyclosporine. FPIA is also used to measure patient serum hormone levels such as the thyroid hormones T3 and T4.

Most existing FPIA assays are competitive binding assays. The molecule being analyzed is called the “analyte”. The fluorescent dye conjugate of antigen molecule is called the “tracer” or “antigen-tracer”. The antibody and tracer are provided and can combine to produce polarized fluorescence when struck with the polarized incident light. The analyte to be measured competes for antibody binding with the tracer, reducing the amount of polarized light emitted. The FPIA reading is inversely proportional to the amount of the antigen (i.e., analyte) in the test sample. In other words, the greater the amount of antigen in the test sample, the lower the FP reading. The dynamic range of the linear relationship between the FP reading and the antigen concentration is narrow. Thus, the resolution of the FPIA assay is low when the amount of the antigen molecule to be measured is either high or low. Furthermore, existing FPIA requires a specific fluorescence labeled molecule for each assay of a particular antigen molecule. This increases the costs of the assays and requires a significant amount of time to set up the assays. In a high throughput scenario, it also increases the possibility of cross-contamination.

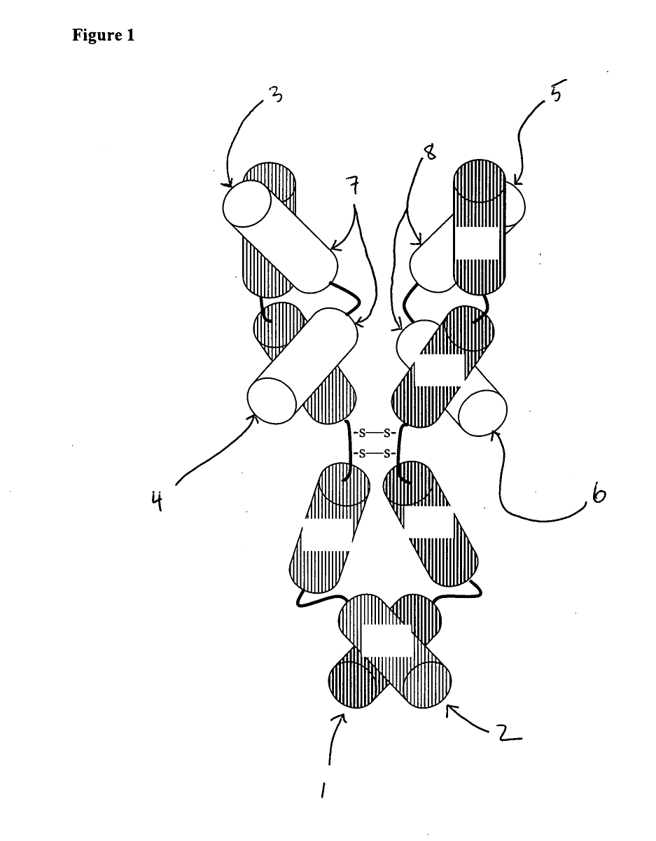

The terms immunoglobulins and antibodies as used herein refer to the protein molecules secreted by immune B cells to defend human and animal bodies against external assaults that include, but are not limited to, non-self proteins, DNA's, and pathogens such as bacteria and viruses. Each antibody (i.e., immunoglobulin, See FIG. 1) consists of four polypeptides, two heavy chains (See FIG. 1, 1 and 2) and two light chains (See FIG. 1, 7 and 8), joined to form a “Y” shaped molecule. The amino acids of N-terminal halves of the arms of the “Y” are the variable regions (See FIG. 1, 3 and 5). The amino acid sequences of variable regions have great diversity so that antibodies can be made to recognize any and every antigen the body encounters. The amino acids of C-terminal halves of the arms and those of the stem region of the “Y” form the constant region. The constant region has many biological functions including triggering the complement reactions to destroy the antigen. Constant region also defines the class (or isotype) of an antibody. Antibodies are divided into five major classes, IgM, IgG, IgA, IgD and IgE, based on their heavy chain constant region structures.

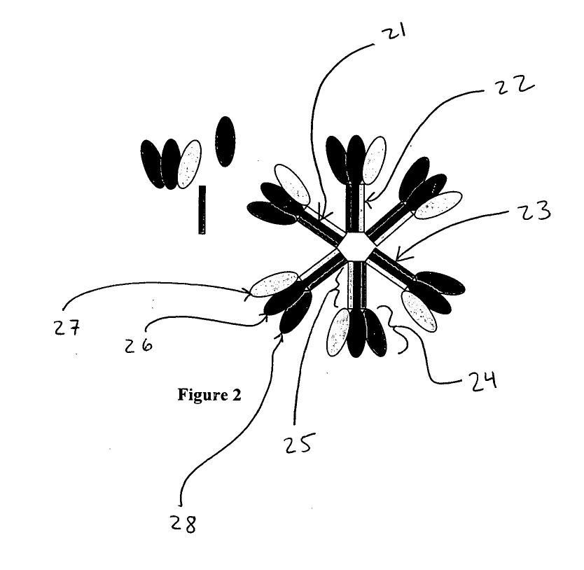

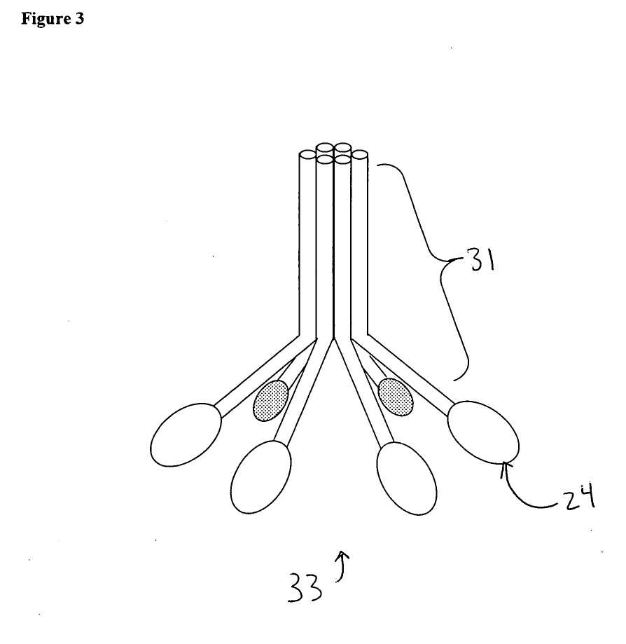

In an exemplary embodiment, the present invention is directed towards a category of low molecular weight omni-bio-tracer. The omni-bio-tracers are polypeptides or non-polypeptides derived from a subunit, C1q, of first complement component, C1. See FIGS. 2 and 3. This omni-bio-tracer is applicable to nearly all assays involving detection of immune complexes, thus the term ‘omni’.

C1q is a large protein (molecular weight of 459.3 kDa, See FIG. 2) consisting of six chains each of A chains (See FIG. 2, 21), B chains (See FIG. 2, 22), and C chains (See FIG. 2, 23). Each chain consists of approximately 225 amino acid residues. See SEQ. I.D. Nos. 2, 3, and 4 respectively. Each of the A, B and C chain has four cysteine residues at positions 4, 135, 154, and 171 (with reference to the numbering of the B chain amino acids). The position numbering is the standard method from the N-terminal to the C-terminal end. One A and one B chain form an inter-chain disulfide bond by the cysteine residues at position 4, while two C chains form an inter-chain disulfide bond by this cysteine residues. The three cysteine residues (positions 135, 154, and 171) each produce one intra-chain disulfide bond and one free thiol group per gC1q domain.

Position 4 is very close to the N-terminal end. It will be eliminated by enzyme digestion of the collagen like region (CLR). Therefore, the three chains of the remaining globular heads will be held together by non-covalent bonds such as hydrogen bonds, hydrophobic interactions, ionic bonds or other weak molecular interactions.

Approximately 135 residues of the C-terminal portion of these three chains form the ‘globular head’ of C1q (See FIG. 2, 24). See FIGS. 2 and 3, and SEQ. I.D. No. 1. This globular head (gC1q) is responsible for the high affinity binding to the C1q-specific binding region of the constant region of certain classes of immunoglobulins, such as IgM and some subclasses of IgG. The interaction between C1q and the antibody-antigen complexes is specific and is independent of the binding specificity between the antibodies and their corresponding antigens. This forms the basis of omni-bio-tracer where one C1q-based tracer is suitable for detecting nearly all antibody-antigen complexes.

Omni-bio-tracer based on C1q circumvents the aforementioned drawbacks of FPIA that are based on competition between the antigen tracer and the analyte. The assays using this omni-bio-tracer are not competitive assays and the degree of fluorescence polarization is directly, positively proportional to the amount of immune complexes formed by antibody and analyte. Therefore, it provides high sensitivity and resolution in a wide detecting region of analyte concentration.

Studies of recombinant forms of the globular head region suggest that each globular head of C1q is composed of three structurally and functionally independent domains/modules. The heterotrimeric organization thus could offer functional flexibility and versatility to the whole C1q molecule.

The C-terminal fragments of the A, B, and C chains of C1q are genetically, structurally, and functionally independent modules. The recombinant forms of the C1q globular head fragment of A, B, and C chains, named as gaC1q, gbC1q, and gcC1q respectively, can bind to the C1q binding site on the constant region of aggregated IgG and/or IgM. The definition of the gaC1q, gbC1q, and gcC1q, in terms of amino acid sequences, is described in Section 2.

Human IgM, IgG1, and IgG3 as well as mouse IgG2a and IgG2b can bind C1q with high affinity. Experiments with mouse IgG2b mutants have revealed that Glu-318-X-Lys320-X-Lys322 is a common core motif on the constant region of the immunoglobulin molecule for C1q binding. The term C1q binding site as used herein, refers to a core polypeptide motif on the constant region of immunoglobulins that bind C1q. The motif includes, but is not limited to Glu-X-Lys-X-Lys, where X can be any amino acid.

Fluorescence polarization assays can be used to identify therapeutic agents and targets of therapeutic agents. The term ‘identifying’ as used herein also includes profiling, detecting, and discovering. Profiling as used herein refers to the analyses of the total cellular protein expression patterns, kinds of proteins expressed in the cells or tissues, and differences in the former two between normal and disease tissues. The technologies used for protein expression profiling include protein arrays (or protein chips), 2-dimensional gel electrophoresis, high-throughput yeast two-hybrid approaches and analysis of protein complexes using affinity tag purification. The term ‘therapeutic agent’ as used herein can refer to many different compounds including, but not limited to inorganic chemical compounds, organic chemical compounds, proteins, polypeptides, and antibodies. Fluorescence polarization assays can also be used to detect microbial pathogens in water, soil or air. The term ‘pathogens’ as used herein refers to all microorganisms that could potentially cause human and animal diseases. Examples include, but are not limited to, protozoa, fungi, bacteria, viruses, and prions. One possible immediate application of the present invention is for a group of pathogens that include the human immunodeficiency viruses, mycobacterium tuberculosis, the Ebola virus, the Hepatitis B, C, or D viruses, small pox virus, and the anthrax bacteria. The term ‘pathogens’ as used herein also includes various strains and mutations thereof.

Test samples for FPIA can include, but are not limited to, animal or plant cells, tissues, body fluids, smears, micro-organism cultures, environmental samples of air, water and soil. Test samples for FPIA can also be components extracted from the aforementioned samples. The antibodies used in the FPIA can be polyclonal antibodies, monoclonal antibodies, recombinant antibodies, and antibody fragments that naturally possess C1q binding ability or that acquire C1q binding ability through genetic modifications.

In a preferred embodiment, the tracer molecules of the present invention emit non-polarized fluorescent light when unbound to an antigen-antibody complex, and polarized fluorescent light when the molecule and the complex are bound to each other. The tracer molecules derived from C1q preferably have a molecular mass in the range of about 0.1-200 kDa. The tracer molecules more preferably have a molecular mass from about 20-100 kDa. These tracer molecules can also be derived from gC1q, gaC1q, gbC1q, and gcC1q. The tracer molecules can contain a conjugate of gaC1q, gbC1q or gcC1q with a fluorescence probe moiety.

The term ‘probe moiety’ as used herein refers to the part of an FP tracer that emits fluorescence when it is stimulated by a light source of a certain wave length. For example, a probe moiety may be a green fluorescence protein, FITC, Texas Red or quantum dots.

The term ‘quantum dots’ as used herein refers to a new class of semiconductor quantum dot fluorescent labels. These labels are applied to biology by conjugation with bio-recognition molecules. These nanometer-sized conjugates are water-soluble and biocompatible. They offer important advantages over organic dyes and lanthanide probes. Specifically, the emission wavelength of quantum-dot nanocrystals can be continuously tuned by changing the particle size. A single light source can be used for simultaneous excitation of all different-sized dots. High-quality quantum dots are also highly stable against photobleaching and have narrow, symmetric emission spectra. These novel optical properties make quantum dots ideal fluorophores for ultrasensitive, multicolor, and multiplexing applications in molecular biotechnology and bioengineering. Quantum dots can also be used as a fluorescent probe moiety in FP, particularly, herein this patent, used with C1q derived molecules to make omni-bio-tracer.

Molecules derived from C1q (i.e., C1q-derived molecules) include polypeptide and non-polypeptide molecules that are based on complement C1q. The C1q-derived polypeptide and non-polypeptide molecules may be produced by the following methods:
1. enzymatically digesting native C1q polypeptide,
2. producing recombinant C1q fragments using genetic engineering technologies,
3. genetically engineering C1q fragments or
4. producing organic or non-organic compounds that functionally mimic C1q polypeptide in its specific binding to immune complexes, and not binding to non-aggregated immunoglobulins.
   1. Enzymatic Digestion of Native C1q Polypeptide:

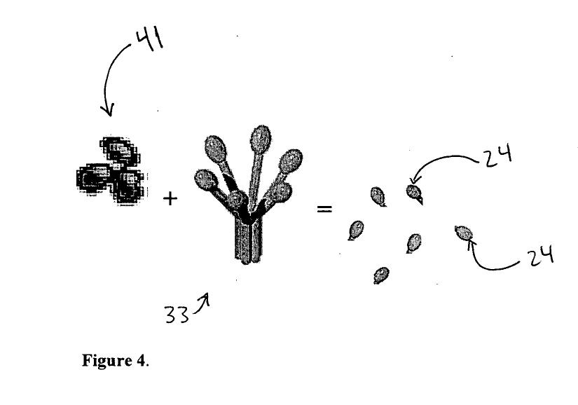

Enzymatic digestion of C1q (See FIG. 4, 33) with proteases(See FIG. 4, 41), including, but not limited to, collagenase, removes the collagen-like domain of natural C1q molecules, but leaves the globular head (gC1q, See FIG. 4, 24) intact. The remaining globular head of the C1q molecule retains the immune complex binding ability. The enzymatically digested C1q molecule and its derivatives have much smaller molecular weights compared to the native C1q molecule. Enzymatically digested C1q and its derivatives also have much smaller molecular weights compared to immune complexes, which generally have molecular weights of more than 140 kDa.

Therefore, when enzymatically digested C1q molecule and its derivatives are labeled with fluorescent molecules to be used as bio-tracer, they bind to immune complexes (antigen-antibody complexes). This binding causes a large change in molecular weight. Once bound to immune complexes, the fluorescence emitted by the labeled C1q derivatives will be polarized due to the great increase in molecular weight.

In an exemplary embodiment of the present invention, small C1q fragments are produced by digesting C1q with proteases to produce the globular head (gC1q) intact with the capacity of binding to immune complexes. An example of one protease that may be used is collagenase. These molecules (i.e., C1q fragments) can be labeled with fluorescent dyes, quantum dots or fluorescent proteins for FP assays using protein-chemical coupling techniques that are already know in the art. Probe-grade pathogen-specific antibodies can be selected by their ability to bind any of the digested C1q molecules tightly and their ability to bind target pathogens.

2. Production of Recombinant C1q Fragments Using Genetic Engineering Technologies:

In addition to the method of enzymatic digestion, smaller fragments of C1q may be produced using recombinant DNA technologies. As discussed above, the globular head of C1q is formed with the C-terminal fragments of the A, B and C chains. The recombinant forms of the C1q globular head fragment of the A, B, and C chains are gaC1q gbC1q and gcC1q, respectively. The ability of gaC1q, gbC1q, and gcC1q to bind aggregated immunoglobulin and the fact that they have relatively small molecular weight (less than 20 kDa) make these chains useful as FP tracers for detecting immune complexes. The term ‘aggregated immunoglobulin’ or ‘aggregated antibodies’ as used herein, refers to antibodies that are bound to antigens. Aggregated antibodies can also refer to antibodies aggregated by heat treatment and are used as calibration agents in immune complex assays. The terms ‘non-aggregated antibodies’ and ‘free antibodies’ as used herein refers to antibodies that are not bound to antigens. Recombinant A, B or C chain globular heads expressed by E. coli are fully functional. It is possible to produce recombinant gaC1q, gbC1q, and gcC1q in large quantities with high purity.

In an exemplary embodiment of the present invention, recombinant vertebrate C1q fragments are produced using recombinant DNA technology that include, but are not limited to the molecules described below. The molecules below are based on human C1q amino acid sequences. C1q amino acid sequences from other animals may be slightly different. The recombinant molecules can be any molecule structurally or functionally similar to gaC1q, gbC1q or gcC1q. See SEQ I.D. No. 1. Molecules are structurally similar to C1q by retaining the minimal critical motifs or amino acid residues of C1q that are required for binding to immune complexes. Molecules are functionally similar to C1q by virtue of their ability to distinguish between immune complexes and unbound immunoglobulins. Thus, like the globular head fragments of C1q, these molecules can bind to immune complexes, but do not bind to unbound immunoglobulins. These molecules are labeled with fluorescent molecules or quantum dots for FP assays using protein-chemical coupling techniques, or labeled with fluorescent proteins by protein coupling chemistry, or by in-frame fusion with fluorescent proteins using cloning techniques.
i. gaC1q—one example of gaC1q is the fragment of human C1q having amino acid residues # 85 to #223 of human C1qA (AAH30153, GI: 20988805). See SEQ I.D. 2. This excludes 81 amino acid residues of the collagen like region (CLR) and the residues N-terminal to CLR. C1q from other sources, such as animals other than humans, may be slightly different, but may also be used.
ii. gbC1q—one example of gbC1q is the fragment of human C1q having amino acid residues #81 to #226 of human C1qB (NP000482, GI: 11038662). See SEQ I.D. 3. This excludes 81 amino acid residues of the collagen like region (CLR) and the residues N-terminal to CLR. C1q from other sources, such as animals other than humans, may be slightly different, but may also be used.
iii. gcC1q—one example of gcC1q is the fragment of human C1q having amino acid residues #78 to #217 of human C1qC (P02747, GI: 20178281). See SEQ I.D. 4. This excludes 81 amino acid residues of the collagen like region (CLR) and the residues N-terminal to CLR. C1q from other sources, such as animals other than humans, may be slightly different, but may also be used.
iv. linked fragments of gaC1q, gbC1q, and/or gcC1q—two or more fragments of gaC1q, gbC1q, and/or gcC1q can be connected by linking amino acid regions to form a single polypeptide chain. Each linking region can range from about 1-280 amino acids. The linked fragments preferably have a total molecular weight of less than about 65 kDa. A polypeptide chain of the linked fragments can include identical fragments (e.g., three fragments of gaC1q and linking regions).
   3. Genetically Engineering C1q Molecules

Recombinant C1q polypeptides with properly engineered mutations can provide higher binding affinity and specificity than the native C1q polypeptide. Specifically, C1q can be mutated using methods that include but not limited to:
i. Mutating gC1q, gaC1q, gbC1q or gcC1q, by random mutations of all amino acids using degenerate polymerase chain reactions, followed by selecting the highest affinity recombinant gC1q.
ii. Identifying canonical amino acids that affect binding affinity of gC1q, gaC1q, gbC1q or gcC1q, to immune complexes using alanine scanning, followed by systematic mutation of those amino acids into all 20 different amino acid choices for selecting the highest affinity recombinant gC1q.
iii. Mutating gC1q, gaC1q, gbC1q or gcC1q, by gene shuffling procedures to further improve the specificity and affinity of its binding to immune complex.
   4. Production of Organic or Non-Organic Compounds That Functionally Mimic C1q Polypeptide in its Binding to Immune Complexes:

As discussed above, human IgM, IgG1 and IgG3 as well as mouse IgG2a, IgG2b have been found to bind C1q with high affinity. Experiments with mouse IgG2b mutants have revealed that Glu-318-X-Lys320-X-Lys322 is a common core motif on the constant region of the immunoglobulin molecule for C1q binding.

In an exemplary embodiment of the present invention, chemical compounds can be identified that bind this core motif specifically. These non-polypeptide chemical compounds that are mimetics of C1q, gC1q, gaC1q, gbC1q, and gcC1q, may also be used as FP tracers to detect immune complexes. The term ‘mimetic’ as used herein, includes non-polypeptide chemical compounds that mimic a molecule in its ability to bind specifically to immune complexes, but not to non-aggregated immunoglobulins. Specifically, chemical compounds that bind the Glu-X-Lys-X-Lys motif on immunoglobulins, where X is any amino acid, are candidates to specifically bind immune complexes with high affinity (i.e., aggregated immunoglobulins), but not to non-aggregated immunoglobulins. All of the aforementioned non-polypeptide molecules are candidates for FP tracers that are smaller and more stable than the native C1q and its derivatives. Specifically, compounds can be screened that bind immune complexes, but do not bind free immunoglobulins using methods that include but are not limited to:
i. Screening organic chemical compound libraries for compounds that bind specifically to immune complexes but not to non-aggregated immunoglobulins. One example is to screen organic combinatorial chemical compound libraries for compounds that bind immunoglobulins in immune complexes.
ii. Screening inorganic combinatorial chemical compounds that bind specifically to immune complex but not to non-aggregated immunoglobulins. One example is to screen inorganic chemical compounds to bind the Glu-X-Lys-X-Lys motifs on immunoglobulins in immune complex, where X is any amino acid.
   5. Production of Polypeptides That Gain the Ability to Bind C1q

Many types of polypeptides can bind C1q with high affinity. As discussed above, examples of these polypeptides include human IgM, IgG1 and IgG3 as well as mouse IgG2a, IgG2b. Experiments with mouse IgG2b mutants have revealed that Glu-318-X-Lys320-X-Lys322 is a common core motif on the constant region of the immunoglobulin molecule for C1q binding.

In an exemplary embodiment of the present invention, polypeptides, including some classes of immunoglobulins, that do not naturally bind C1q can be engineered to bind C1q with high affinities. These polypeptides may also be used as FP tracers to detect immune complexes. Specifically, the C1q binding motif, Glu-X-Lys-X-Lys, found on certain immunoglobulins, where X is any amino acid, can be added by genetic engineering to any polypeptides. These engineered polypeptides can then bind C1q with high affinity when they bind to their corresponding partners or binding proteins. For example, an immunoglobulin that does not naturally bind C1q or binds C1q with low affinity can be engineered to have the Glu-X-Lys-X-Lys motif on their Fc or Fab portions. Even a single chain antibody can be added to the Glu-X-Lys-X-Lys motif and become recognizable by C1q once it binds the antigen. When these newly engineered immunoglobulin bind to their corresponding antigen, they will be recognized and detected by C1q. For example, more than one C1q binding motif, Glu-X-Lys-X-Lys, can be added to a polypeptide. For example, the C1q binding motif, Glu-X-Lys-X-Lys, can be chemically coupled to deoxyribonucleotides or ribonucleotides so that the final products can be recognized by C1q when they bind to corresponding polypeptide binding partners. All of the aforementioned polypeptide and derivative molecules are candidates for FP tracers.

It should be emphasized that the description and examples herein have been presented for purpose of providing a clear understanding of the invention. The description is not intended to be exhaustive or to limit the invention to the precise examples disclosed. Many features, advantages, and objects of the present invention will become apparent to one with skill in the art, upon examination of the detailed description. It is intended that all such features, advantages, and objects be included within the scope of the present invention. Furthermore, obvious modifications or variations by one with skill in the art are possible in light of the above teachings without departing from the spirit and principles of the invention. All such modifications and variations are intended to be included within the scope of the present invention.

### EXAMPLES

The present invention is illustrated by the following examples that should not be considered limiting.

### Example 1

Collagenase-Digestion of C1q Molecule

Human C1q is to be incubated with collagenase (type VII, high purity, Sigma) in 37° C. for 3 hours in a digestion buffer consists of 0.05 M Tris/HCl, pH 7.4, 5 mM CaCl₂, and 0.25 mM N-ethylmaleimide. The digestion mixture is then passed through a gel filtration column. The fraction that contains the C1q globulin heads is collected and examined with SDS-PAGE (sodium dodecyl sulfate-polyacrylamide gel electrophoresis) and Western blotting to confirm the molecular weight and the identity of the C1q globular head.

The ability of the newly digested C1q globular heads to bind immune complexes is to be confirmed by ELISA using immobilized or heat aggregated IgG2 as a positive test and IgG4 as a negative test.

### Example 2

Fluorescent Dye Labeling of gC1q

Fluorescent dyes and quantum dots can be conjugated to collagenase digested C1q globular head and the gaC1q and gbC1 heads by standard amine chemistry.

### Example 3

### Fluorescent Dye Labeling of gaC1q and gbC1q

Fluorescent dyes and quantum dots can be conjugated to collagenase digested C1q globular head and the gaC1q and gbC1 heads by standard amine chemistry.

### Example 4

Green Fluorescent Protein (GFP)-gaC1q and gbC1q Conjugates

GFP conjugates of gaC1q and gbC1q are made by cloning gaC1q and gbC1q coding sequences in frame with GFP into a GFP expression vector using standard cloning methods.

### Example 5

Screening for Non-Polypeptide Chemical Derivatives of C1q

Organic compound libraries and/or combinatorial chemical libraries are screened with immune complexes to find those compounds that compete specifically with C1q binding.

### Example 6

Recombinant gaC1q and gbC1q

Recombinant gaC1q, gbC1q are made by conventional gene cloning methods and expressed with either prokaryote or eukaryote recombinant protein expression systems.

### Example 7

Introduction of Mutations into gaC1q and gbC1q

To improve their affinities and specificities, gaC1q and gbC1q are mutated by methods including, but not limited to, point mutations, in vitro evolution, or gene shuffling methods.

### Example 8

Probe-Grade Pathogen-Specific Antibodies

Monoclonal antibodies against a variety of antigens are raised with corresponding microorganisms or their immunogenic antigens. Mouse myeloma cells that produce IgG2a or IgG2b, or human cells that produce IgM, IgG3 or IgG1 are chosen as the hybridoma partners for strong binding of the immunoglobulins with C1q after aggregation. Monoclonal antibodies produced as described that bind both the pathogen and C1q with high affinities are selected and purified with protein A/G affinity chromatography.

### Example 9

Fluorescence Polarization Immunoassays Using C1q-Derived Polypeptide or Non-Polypeptide Tracer Molecules

In accordance with the method of the present invention, test samples that are suspected of containing pathogens are mixed with probe-ready, pathogen specific antibodies, or with a mixture of such antibodies against an array of microbials, together with fluorescence labeled C1q-derived polypeptide or non-polypeptide mimetics to conduct an FP assay. Such test samples include, but not limited to, an environment sample of water or of air, or a surface smear. Polarization angles, fluorescent density and fluorescence half-life are measured for the test samples and are compared with those of controls.

Seq.I.D.No.1

| | |
|---|---|
| | 90 100 109 118 128 138 148 |
| | 92 102 112 122 132 142 152 |
| | 89 99 109 119 CHO 129 139 149 |
| | \| \| \| \| \| \| \| \| |
| Clq_a | QPRPAFSAIRRN--PPMGGNVVIPDTVITNQEEPYQNHSGRFVCTVPGYYYFTFQVLSQWEICLSIVSSS |
| Clq_b | TQKIAFSATRTINVPLRRDQTIRFDHVITNMNNNYEPRSGKFTCKVPGLYYFTYHASSRGNLCVNLMRG- |
| Clq_c | KFQSVFTVTRQTHQPPAPHSLIRFNAVLTNPQGDYDTSTGKFTCKVPGLYYFVYHASHTANLCVLLYRS- |
| | 159 168 178 188 198 208 218 |
| | 162 171 180 190 200 210 219 |
| | 158 165 174 184 194 204 213 |
| | \| \| \| \| \| \| \| |
| Clq_a | RGQVRRSLGFCDTTNKGLFQVVSGGMVLQLQQGDQVWVEKDPKKGHIYQGSEADSVFSGFLIFPSA |
| Clq_b | RERAQKVVTFCDYAYN-TFQVTTGGMVLKLEQGENVFLQATDKNSLLGMEG-ANSIFSGFLLEPDMEA |
| Clq_c | -G--VKVVTFCGHTSK-TNQVNSGGVLLRLQVGEEVWLAVNDYYDMVGIQG-SDSVFSGFLLFPD |

Amino acid sequence of gClq domains aligned with structural similarity.

Amino acid sequence of C1qA

```
Complement component 1, q subcomponent, alpha polypeptide precursor
ACCESSION AAH30153 GI:20988805
Seq.I.D.No.2
    1 MEGPRGWLVL CVLAISLASM VTEDLCRAPD GKKGEAGRPG RRGRPGLKGE QGEPGAPGIR
  61 TGIQGLKGDQ GEPGPSGNPG KVGYPGPSGP LGARGIPGIK GTKGSPGNIK DQPRPAFSAI
121 RRIPPMGGNV VIFDTVITNQ EEPYQNHSGR FVCTVPGYYY FTFQVLSQWE ICLSIVSSSR
181 GQVRRSLGFC DTTNKGLFQV VSGGMVLQLQ QGDQVWVEKD PKKGHIYQGS EADSVFSGFL
241 IFPSA
```

Amino acid sequence of C1qB

```
complement component 1, q subcomponent, beta polypeptide precursor
ACCESSION NP_000482 GI:11038662
Seq.I.D.No.3
    1 MMMKIPWGSI PVLMLLLLLG LIDISQAQLS CTGPPAIPGI PGIPGTPGPD GQPGTPGIKG
  61 EKGLPGLAGD HGEFGEKGDP GIPGNPGKVG PKGPMGPKGG PGAPGAPGPK GESGDYKATQ
121 KIAFSATRTI NVPLRRDQTI RFDHVITNMN NNYEPRSGKF TCKVPGLYYF TYHASSRGNL
181 CVNLMRGRER AQKVVTFCDY AYNTFQVTTG GMVLKLEQGE NVFLQATDKN SLLGMEGANS
241 IFSGFLLFPD MEA
```

Amino acid sequence of C1qC

```
Complement Clq subcomponent, C chain precursor.
ACCESSION P02747 GI:20178281
Seq.I.D.No.4
    1 MMMKIPWGSI PVLILLLLLG LIDISQAQLS CTGPPAIPGI PGIPGTPGPD GQPGTPGIKG
  61 EKGLPGLAGD HGEFGEKGDP GIPGNPGKVG PKGPMGPKGG PGAPGAPGPK GESGDYKATQ
121 KIAFSATRTI NVPLRRDQTI RFDHVITNMN NNYEPRSGKF TCKVPGLYYF TYHASSRGNL
181 CVNLMRGRER AQKVVTFCDY AYNTFQVTTG GMVLKLEQGE NVFLQATDKN SLLGMEGANS
241 IFSGFLLFPD MEA
```

1. A method of identifying a polypeptide using a C1q derived molecule as a tracer molecule in fluorescence polarization.

2. The method of claim 1, wherein said tracer molecule is gC1q.

3. The method of claim 1, wherein said tracer molecule is gaC1q.

4. The method of claim 1, wherein said tracer molecule is gbC1q.

5. The method of claim 1, wherein said tracer molecule is gcC1q.

6. The method of claim 1, wherein said tracer molecule is any combination of gC1q, gaC1q, gbC1q or gcC1q, and wherein said molecule is less than about 65 kDa.

7. The method of any one of claims 1-6, wherein said polypeptide is an immune complex.

8. A method of identifying a polypeptide using a recombinant molecule as a tracer molecule in fluorescence polarization, wherein said molecule is structurally or functionally similar to the C1q A chain (Seq. I.D. No. 2).

9. A method of identifying a polypeptide using a recombinant molecule as a tracer molecule in fluorescence polarization, wherein said molecule is structurally or functionally similar to the C1q B chain (Seq. I.D. No. 3).

10. A method of identifying a polypeptide using a recombinant molecule as a tracer molecule in fluorescence polarization, wherein said molecule is structurally or functionally similar to the C1q C chain (Seq. I.D. No. 4).

11. A method of identifying a polypeptide using a recombinant molecule as a tracer molecule in fluorescence polarization, wherein said tracer molecule is a combination of molecules that are structurally or functionally similar to the C1q A, B or C chains (Seq. I.D. No. 2, 3 or 4).

12. The method of any one of claims 8-11 wherein said polypeptide is an immune complex.

13. A molecule which can be used as a tracer molecule in fluorescence polarization, wherein said molecule is genetically engineered from the globular head of C1q to have a higher binding affinity to a Glu-X-Lys-X-Lys motif than said globular head before said genetic engineering, wherein X is an amino acid.

14. A molecule which can be used as a tracer molecule in fluorescence polarization, wherein said molecule is genetically engineered from a C1q fragment chosen from the group consisting of gaC1q, gbC1q or gcC1q, to have a higher binding affinity to a Glu-X-Lys-X-Lys motif than the C1q fragment before said genetic engineering, wherein X is an amino acid.

15. A polypeptide genetically engineered to include a Glu-X-Lys-X-Lys motif, wherein X is an amino acid and, said polypetide emits non-polarized fluorescent light when unbound to tracer molecule, and said molecule and said polypeptide emit polarized fluorescent light when bound to each other.

16. A polypeptide genetically engineered to include a Glu-X-Lys-X-Lys motif, wherein X is an amino acid and, said polypetide emits non-polarized fluorescent light when unbound to a C1q derived molecule, and said molecule and said polypeptide emit polarized fluorescent light when bound to each other.

17. A method of identifying a polypeptide comprising using a non-polypeptide chemical compound that binds a Glu-X-Lys-X-Lys motif as a tracer molecule in fluorescence polarization.

18. A molecule comprising a non-polypeptide compound which binds the core motif of the Fc region of an immunoglobulin wherein said molecule emits non-polarized fluorescent light when unbound to an antigen-antibody complex and emits polarized fluorescent light when bound to an antigen-antibody complex.

19. A method of producing recombinant C1q fragments comprising cloning of C1q coding sequences into expression vectors and the expression of C1q recombinant proteins using such vectors in prokaryotic or eukaryotic cells, wherein said fragment emits non-polarized fluorescent light.


SEQUENCE LISTING (3 sequences)

SEQ ID NO: 1 - protein, 245 aa, Homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 253 aa, Homo sapiens
MMMKIPWGSIPVLILLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 3 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD